(19) United States
(12) Patent Application Publication
(10) Pub. No.: US 2014/0356860 A1
(43) Pub. Date: Dec. 4, 2014

(54) METHOD FOR ISOLATING NUCLEIC ACIDS FROM A VETERINARY WHOLE BLOOD SAMPLE

(71) Applicants: Denis Flügge, Hilden (DE); Lillian Krüger, Hilden (DE); Mario Scherer, Hilden (DE)

(72) Inventors: Denis Flügge, Hilden (DE); Lillian Krüger, Hilden (DE); Mario Scherer, Hilden (DE)

(73) Assignee: QIAGEN GMBH, Hilden (DE)

(21) Appl. No.: 14/344,433

(22) PCT Filed: Sep. 13, 2011
(86) PCT No.: PCT/EP2011/065827

§ 371 Date: Aug. 20, 2014

(51) Int. Cl. C12Q 1/68; C12Q 1/70

(52) U.S. Cl. CPC C12Q 1/6806; C12Q 1/70
USPC 435/5; 536/25.4

(57) ABSTRACT

The present invention provides a method for isolating nucleic acids from a veterinary whole blood sample, said method comprising at least the following steps a) preparing a binding mixture comprising—the lysed sample—at least one chaotropic agent—at least one alcohol—at least one polyoxyethylene fatty alcohol ether; b) passing the binding mixture through a column comprising a nucleic acid binding solid phase thereby binding the nucleic acids to the nucleic acid binding solid phase; c) optionally washing the nucleic acids while being bound to the solid phase; d) optionally eluting the nucleic acids from the solid phase. It was found that the addition of the specific non-ionic detergent overcomes the problems of the prior art methods, wherein the column clogs what prevents the efficient nucleic acid isolation from this difficult sample. When the specific non-ionic detergent is included into the binding mixture, no clogging occurs thereby allowing the efficient isolation of nucleic acids from veterinary whole blood samples.

### FIELD OF THE INVENTION

The present invention pertains to a method for isolating nucleic acids from veterinary whole blood samples. In particular, it provides a method for isolating pathogen nucleic acids from veterinary whole blood samples.

### BACKGROUND OF THE INVENTION

Numerous methods for isolating nucleic acids from different samples are known in the prior art. However, the respective methods do not work equally well for all samples. E.g., nucleic acids can be efficiently isolated from certain samples such as tissue, plasma, serum or urine when using one method. However, said method might fail to efficiently isolate nucleic acids from other samples such as whole blood samples. In particular biological samples containing high cell numbers, protein and/or lipid content, such as veterinary whole blood samples obtained from large domestic animals such as horse or cattle, pose a particular challenge. The methods known in the prior art are often not satisfactory in efficiently isolating nucleic acids from respective difficult samples. For example, current spin column based nucleic acid isolation methods have only a very limited suitability for processing veterinary whole blood samples, particularly blood samples obtained from large domestic animals such as cattle, sheep, goat, horse and pig. The major problem when using established nucleic acid isolation methods for isolating nucleic acids from respective animal whole blood samples is that the spin columns easily clog due to the special composition of these samples. In particular high cell numbers, large amount of proteins and/or lipids can be causative for this clogging effect. The columns are obstructed with blood derived substances, resulting in a low purification efficiency. As a result, less or even no nucleic acids are isolated. Furthermore, the isolated nucleic acids are often contaminated with impurities/inhibitory substances, which impair the downstream performance of the isolated nucleic acids, in particular in amplification reactions such as RT-PCR. This renders the detection of a certain target nucleic acid in the isolated nucleic acids difficult. Furthermore, the observed clogging effect results in that only a small amount of sample can be processed in one sample preparation. Usually, the sample input volume must be limited in order to reduce the clogging (for example to 25 μl to 50 μl instead of for example 200 μl).

The described clogging in particular poses a problem, when intending to isolate nucleic acids from a large amount of samples using for example a 96 multi format or when using automated systems. Here, it is rather impossible to individually solve the clogging problem when occurring with a specific sample (for example by diluting the binding mixture, re-application of the binding mixture to the membrane and/or application of the binding mixture to a different membrane). Therefore, in particular when processing a larger number of samples and/or when using an automated system, the respective nucleic acid sample is lost for analysis. This is unacceptable in the medical and/or diagnostic field as the loss of the sample would make a recollection of the sample necessary. Thus, in particular in the veterinary diagnostic and medical field, a reliable, efficient method is needed that is also suitable for automation and allows to isolate nucleic acids from veterinary whole blood samples. The described clogging problems in particular occur when using a nucleic acid isolation chemistry that is based on the use of chaotropic agents, detergents and alcohol in conjunction with a column. However, spin column based isolation methods have their advantages with respect to the obtainable nucleic acid yield and the ease of handling, and are therefore often preferred.

The low purification efficiency and the limitation with respect to the possible input volume are significant drawbacks when intending to isolate specific target nucleic acids that may be comprised in the whole blood sample. E.g. pathogen nucleic acids such as for example viral and/or bacterial nucleic acids may only be comprised in the whole blood samples in low amounts. The observed low purification efficiency and the limitations with respect to the input volume result in that regular nucleic acid isolation methods often do not allow to isolate the target pathogen nucleic acids in sufficient amounts in order to allow the subsequent performance of a standard detection assay such as for example a polymerase chain reaction. However, the detection of respective pathogen nucleic acids in animal whole blood samples is one of the major reasons for isolating nucleic acids from respective samples in the first place. Therefore, there is a particular need in the prior art to provide a nucleic acid isolation method that allows to isolate nucleic acids with high yield and purity from animal whole blood samples. Furthermore, there is a need to provide a nucleic acid isolation method that allows to isolate nucleic acids from a broad variety of animal samples, including whole blood samples.

Therefore, it is the object of the present invention to provide an improved column based nucleic acid isolation protocol that allows to isolate nucleic acids from veterinary whole blood samples.

### SUMMARY OF THE INVENTION

The present invention is based on the finding that the clogging of the column can be prevented if a specific non-ionic detergent is added to the binding mixture. The inventors found that the addition of a polyoxyethylene fatty alcohol ether prevents the clogging that is observed with the prior art isolation methods that do not incorporate a respective detergent. Therefore, the present invention provides a solution to the problem that was known in the prior art. It was highly unexpected that the clogging of the column can be prevented when adding a specific non-ionic detergent to the binding mixture, because the observed clogging effect occurs when the binding mixture comprises other non-ionic detergents, such as for example the commonly used TritonX-100 or Tween 20. Therefore, it was highly surprising that the choice of the detergent—in particular within the group of non-ionic detergents—would have such a strong impact on the clogging of the membrane and the efficiency of the nucleic acid isolation. As is shown by the examples, the method according to the present invention allows to isolate nucleic acids comprised in veterinary whole blood samples with high efficiency, purity and reliability. In particular, said method allows the efficient isolation of pathogen nucleic acids from veterinary whole blood samples. This is a considerable improvement over prior art methods, which even though allowing to efficiently isolate nucleic acids from other veterinary samples such as plasma, urine or tissue do not allow to efficiently isolate nucleic acids and in particular pathogen nucleic acids from veterinary whole blood samples.

According to a first aspect, the present invention provides a method for isolating nucleic acids from a veterinary whole blood sample, wherein said method comprises at least the following steps:
a) preparing a binding mixture comprising
   the lysed sample
   at least one chaotropic agent
   at least one alcohol
   at least one polyoxyethylene fatty alcohol ether;
b) passing the binding mixture through a column comprising a nucleic acid binding solid phase thereby binding the nucleic acids to the nucleic acid binding solid phase;
c) optionally washing the nucleic acids while being bound to the solid phase;
d) optionally eluting the nucleic acids from the solid phase.

As discussed above, it was found that the incorporation of at least one polyoxyethylene fatty alcohol ether in the binding mixture enables the passage of the binding mixture through the nucleic acid binding solid phase comprised in the column. The clogging of the column that is observed with the prior art methods is thereby prevented, thereby allowing the efficient, undisturbed isolation of nucleic acids from a veterinary whole blood sample. The method is thus very reliable.

According to a further aspect, a method for isolating pathogen nucleic acids from a veterinary whole blood sample is provided, wherein the method according to the first aspect of the present invention is performed. As is shown by the examples, the method according to the present invention particularly allows to isolate pathogen nucleic acids with high efficiency and reliability from veterinary whole blood samples. This advantage is particularly important for the medical and/or diagnostic field wherein veterinary blood samples are analysed for the presence or absence of specific pathogens.

### BRIEF DESCRIPTION OF THE FIGURES

FIG. 1: shows the clogging of bovine whole blood with a standard method employing a binding mixture containing guanidine hydrochloride in combination with Tween 20 (tube 1+2). No clogging occurs when using a binding mixture containing polyoxyethylene(20) cetyl ether (tubes 3+4).

FIG. 2: shows the results of a BVDV detection assay using nucleic acids that were isolated using different lysis/binding conditions of the method according to the present invention in comparison to a method which does not incorporate polyoxyethylene(20) cetyl ether but Triton X-100 as detergent. The lower Ct values that are obtained with the method according to the present invention demonstrate that said method is more suitable to isolate nucleic acids from different veterinary whole blood samples.

FIG. 3: shows the results of a BVDV detection assay using nucleic acids that were isolated using different lysis/binding conditions of the method according to the present invention in comparison to a method using Tween 20 as detergent. The lower Ct values that are obtained with the method according to the present invention demonstrate that said method is more suitable to isolate nucleic acids from different veterinary whole blood samples.

FIG. 4: shows the results of a BHV1 detection assay using nucleic acids that were isolated using different lysis/binding conditions of the method according to the present invention. The figure indicates the type of sample and the concentration of polyoxyethylene(20) cetyl ether.

FIGS. 5 and 6: show a photometric measurement and an ethidium bromide stained agarose gel of 6 different animal blood samples processed with protocol 2. As marker, a lambda DNA/Hind III marker was used.

FIG. 7: shows that equal results are obtained when different users isolate nucleic acids with the method according to the present invention.

### DETAILED DESCRIPTION OF THE INVENTION

According to the first aspect, the present invention provides a method for isolating nucleic acids from a veterinary whole blood sample, wherein said method comprises at least the following steps:
a) preparing a binding mixture comprising
   the lysed sample
   at least one chaotropic agent
   at least one alcohol
   at least one polyoxyethylene fatty alcohol ether;
b) passing the binding mixture through a column comprising a nucleic acid binding solid phase thereby binding the nucleic acids to the nucleic acid binding solid phase;
c) optionally washing the nucleic acids while being bound to the solid phase;
d) optionally eluting the nucleic acids from the solid phase.

The essence of the present invention lies in the incorporation of a specific detergent in the binding mixture. Said specific detergent surprisingly prevents the clogging of the nucleic acid binding column. This was unexpected because other non-ionic detergents such as Triton X-100 or Tween20 can not prevent the clogging of the column when being used with a similar/identical nucleic acid binding chemistry comprising the use of a chaotropic agent and an alcohol. As is shown by the examples, it is the incorporation of a polyoxyethylene fatty alcohol ether into the binding mixture that prevents the clogging of the column. While very similar nucleic acid isolation protocols which also use chaotropic agents, alcohol and a non-ionic detergent are equally effective as the method according to the present invention to isolate nucleic acids from other veterinary samples, such as for example plasma, serum or urine, they fail to isolate nucleic acids from veterinary whole blood samples. Causative for said problems is apparently the special composition of veterinary whole blood samples, in particular high cell number, protein and/or lipid content. The examples show that the method according to the present invention allows to efficiently isolate nucleic acids from all veterinary samples tested, including veterinary whole blood samples from different species, while other methods fail.

In step a) of the method according to the present invention the binding mixture is prepared. With the preparation of the binding mixture the conditions are established that allow the binding of the nucleic acids comprised in the binding mixture to the nucleic acid binding solid phase in step b). The binding mixture comprises the lysed sample and accordingly, the nucleic acids that were released from the sample, at least one chaotropic agent, at least one alcohol and, this is important, at least one polyoxyethylene fatty alcohol ether.

The term “fatty alcohol” in particular means for the purposes of the present invention alcohols having a chain length of from 6 to 22 carbon atoms, preferably 8 to 20 carbon atoms, preferentially 10 to 18 carbon atoms, particularly preferably 12 to 18 carbon atoms. Preference is in particular given to alcohols having 12, 14, 16 or 18 carbon atoms. Although the fatty alcohols may be mono- or polyunsaturated, they are preferably saturated fatty alcohols.

The term “polyoxyethylene” in particular means for the purposes of the present invention an HO—(CH2CH2O)n unit, with n being preferably an integer from 2 to 150, further preferably from 4 to 120, still further preferably from 8 to 80, and most preferably an integer selected from 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 and 150.

Preferred examples of suitable polyoxyethylene fatty alcohol ethers are polyethoxylated lauryl, cetyl, oleyl, or stearyl alcohols which may be used alone or as mixture. According to a preferred embodiment of the invention, the at least one polyoxyethylene fatty alcohol ether comprises a fatty alcohol component having from 6 to 22 carbon atoms and a polyoxyethylene component having from 2 to 150 (CH2CH2O) units. Preferably, the polyoxyethylene fatty alcohol ether is selected from the group consisting of polyoxyethylene lauryl ether, polyoxyethylene cetyl ether, polyoxyethylene stearyl ether and/or polyoxyethylene oleyl ether. The respective binding mixture can be prepared by various means and preferred embodiments are described in the following.

Lysis of the whole blood sample can be (at least partially) achieved in the binding mixture. However, preferably, the sample is at least partially lysed prior to preparing the binding mixture in order to efficiently release the nucleic acids first. Therefore, according to one embodiment, the preparation of the binding mixture in step a) comprises the following steps:
i) lysing the veterinary whole blood sample; and
ii) adding one or more additives, preferably in form of a binding solution, to the lysed sample, thereby preparing a binding mixture comprising
   the lysed sample
   at least one chaotropic agent
   at least one alcohol
   at least one polyoxyethylene fatty alcohol ether.

Different methods can be used in order to achieve the lysis of the sample and suitable lysis methods are known in the prior art. They may also vary depending on the type of target nucleic acid that is supposed to be isolated from the animal whole blood sample. Generally, the lysis step may include but it is not limited to mechanical, chemical, physical and/or enzymatic actions on the sample. Examples of respective lysis steps include but are not limited to grinding the sample in a bead mill or in the presence of glass beads, the application of ultrasound, heating, the addition of detergents and/or the addition of protein degrading compounds, such as for example protein degrading enzymes or chaotropic agents.

According to one embodiment, the lysis involves the addition of at least one proteolytic enzyme to the veterinary whole blood sample. A proteolytic enzyme refers to an enzyme that catalyzes the cleavage of peptide bounds, for example in proteins, polypeptides, oligopeptides and peptides. Exemplary proteolytic enzymes include but are not limited to proteinases and proteases in particular subtilisins, subtilases, alkaline serine proteases and the like. Subtilases are a family of serine proteases, i.e. enzymes with a serine residue in the active side. Subtilisins are bacterial serine protease that has broad substrate specificities. Subtilisins are relatively resistant to denaturation by chaotropic agents, such as urea and guanidine hydrochloride and anionic detergents such as sodium dodecyl sulfate (SDS). Exemplary subtilisins include but are not limited to proteinase K, proteinase R, proteinase T, subtilisin, subtilisin A, QIAGEN Protease and the like. Discussions of subtilases, subtilisins, proteinase K and other proteases may be found, among other places in Genov et al., Int. J. Peptide Protein Res. 45: 391-400, 1995. Preferably, the proteolytic enzyme is proteinase K. In non-limiting aspects, the proteolytic enzyme is added for lysis to the veterinary whole blood sample in a concentration selected from about 0.1 mg/ml to about 10 mg/ml, from about 0.5 mg/ml to about 5 mg/ml, from about 1 mg/ml to about 4.0 mg/ml, from about 1.5 mg/ml to about 2.5 mg/ml and about 2 mg/ml. The indicated concentration ranges refer to the mixture of the proteolytic enzyme and veterinary whole blood sample.

The whole blood sample, optionally but preferably after the addition of further agents that support the lysis (such as chaotropic agents and/or detergents, see below), is incubated under conditions that allow the digestion of the sample by the proteolytic enzyme for at least 3 min, preferably at least 5 min, more preferred for at least 10 min. The incubation may occur at room temperature (e.g. for 10 to 20 min), what still allows the digestion of the sample but obviates the need for special equipment that enables the heating of the sample. However, the sample may also be incubated under conditions that support the digestion of the sample such as heating and/or shaking. Preferably, the sample is heated to a temperature of at least 35° C., at least 40° C., or at least 50° C. and preferably is heated to a temperature of at least 55° C. during incubation. Using respective higher temperatures during incubation is in particularly favourable if a proteolytic enzyme such as proteinase K is used as protein-degrading compound that shows its optimal, respective highest activity at higher temperatures. Under such conditions, the digestion of the sample is promoted. Of course, a temperature should be used wherein the proteolytic enzyme is active. Furthermore, it is preferred that the said incubation step is performed while agitating the sample. Non-limiting examples of agitation include shaking, stirring, mixing, or vibrating. In certain aspects, agitation comprises shaking. The shaking can be one, two, or three dimensional shaking. A variety of shaking or agitating devices can be used. Non-limiting examples include the Thermomixer (Eppendorf), TurboMix (Scientific Industries), Mo Bio Vortex Adapter (Mo Bio Laboratories), Microtube holder vortex adapter (Troemner), and the Microtube foam rack vortex attachment (Scientific Industries). Agitating can be performed for example in a mixer with at least 50 rpm, at least 100 rpm, at least 200 rpm or at least 500 rpm. Preferably, heating and agitation is simultaneously performed, for example by using a thermomixer or an equivalent apparatus that allows simultaneous heating and agitation. When using at least one proteolytic enzyme, incubation conditions are used that ensure that said enzyme works efficiently and is catalytically active. The conditions depend on the proteolytic enzyme used and are known, respectively determinable by the skilled person. Preferably, the incubation is performed in the presence of salts and/or ions that promote and/or maintain the activity of the proteolytic enzyme. Suitable salts include but are not limited to NaCl, KCl, MgCl₂, or CaCl₂ or chaotropic agents such as chaotropic salts. According to one embodiment, the incubation conditions for lysing the sample with the proteolytic enzyme comprise one or more of the following heating, agitation, the presence of salts, a pH value of between 6 to 9 and/or an incubation period of at least 3 min, preferably at least 5 min, most preferred for at least 10 min.

Preferably, the lysis of the veterinary whole blood sample involves the addition of at least one chaotropic agent. Any chaotropic agent can be used for that purpose that causes disorder in a protein or nucleic acid by, for example, but not limited to altering the secondary, tertiary or quaternary structure of a protein or a nucleic acid. Preferably, a chaotropic salt is used. The chaotropic salt preferably comprises guanidinium, thiocyanate, isothiocyanate, perchlorate, trichloroacetate and/or trifluoroacetate as chaotropic ion. Preferably, the chaotropic agent is selected from the group consisting of guanidinium hydrochloride, guanidinium thiocyanate, guanidinium isothiocyanate, sodium thiocyanate, sodium iodide, sodium perchlorate, sodium trichloroacetate, sodium trifluroacetate and urea. Also a mixture of chaotropic agents can be used. Preferably, guanidinium hydrochloride and/or guanidinium thiocyanate is used as chaotropic agent for lysis. Preferably, the chaotropic agent is contained in a lysis solution such as a lysis buffer that is added to the veterinary whole blood sample. The lysis solution may comprise the chaoptropic agent, which preferably is a chaotropic salt as mentioned above, in a concentration that lies in a range selected from about 0.1M up to the saturation limit, about 0.2M to 6M, about 0.5M to 4M or about 0.5M to 3M. The chaotropic agent that has been added for lysis is accordingly also comprised in the lysed sample. As the lysed sample forms part of the binding mixture, a chaotropic agent that has been added to the sample for lysis also contributes to the overall concentration of chaotropic agent, respectively chaotropic agents, in the binding mixture.

According to a preferred embodiment, the lysis of the sample involves the addition of at least one detergent. The detergent may be an anionic detergent, a cationic detergent and/or a non-ionic detergent. Preferably, at least one non-ionic detergent is added during lysis. The specific non-ionic detergent that is according to the teachings of the present invention included in the binding mixture to prevent the clogging of the column may already be added during lysis of the sample. Therefore, according to one embodiment, at least one polyoxyethylene fatty alcohol ether preferably selected from the group consisting of polyoxyethylene lauryl ether, polyoxyethylene cetyl ether, polyoxyethylene stearyl ether and polyoxyethylene oleyl ether is added to the veterinary whole blood sample to support the lysis. Thereby, the respective detergent is already present in the lysed sample and therefore becomes included in the binding mixture. It is preferred to add more of the respective detergent after lysis of the sample in order to establish the binding conditions and to ensure that the binding mixture comprises the respective polyoxyethylene fatty alcohol ether in a sufficiently high concentration to prevent clogging. According to one embodiment, at least one non-ionic detergent different from a polyoxyethylene fatty alcohol ether or mixtures of respective non-ionic detergents different from a polyoxyethylene fatty alcohol ether are added for lysis. Here, the polyoxyethylene fatty alcohol ether is not added during lysis but afterwards to establish the binding conditions. According to one embodiment lysis involves the addition of at least one non-ionic detergent selected from the group of alkylglucosides and/or polyoxyethylene alkyl phenyl ether. Preferably, the respective non-ionic detergent or mixture of non-ionic detergents is comprised in the lysis mixture in a concentration selected from at least 0.5%, at least 1%, at least 3%, at least 4% and at least 5%. Preferred concentration ranges include but are not limited to 0.5% to 15%, more preferred 1.5% to 10% and 2% to 7%. As alkylglucoside, preferably a non-ionic detergent from the group of the polysorbates, preferably polysorbate 20, polysorbate 40 or polysorbate 80, more preferred polysorbate 20 is used. Preferred examples of polyoxyethylene alkyl phenyl ethers include Triton X-100 and Nonidet P-40. Preferably, Triton X-100 and/or Tween 20 are added to the veterinary whole blood sample to achieve the lysis. The respective detergents may also be included in the lysis solution comprising the chaotropic agent (see above). It is also within the scope of the present invention to add the polyoxyethylene fatty alcohol ether in addition to the at least one non-ionic detergent different from a polyoxyethylene fatty alcohol ether during lysis.

Furthermore, the lysis of the sample may involve the addition of at least one chelating agent. Suitable chelating agents include but are not limited to diethylenetriaminepentaacetic acid (DTPA), ethylenedinitrilotetraacetic acid (EDTA), ethylene glycol tetraacetic acid (EGTA) and N,N-bis(carboxymethyl)glycine (NTA). According to a preferred embodiment, EDTA is used. As used herein, the term “EDTA” indicates inter alia the EDTA portion of an EDTA compound such as, for example, K₂EDTA, K₃EDTA or Na₂EDTA. Using a chelating agent such as EDTA also has the advantageous effect that nucleases such as DNases are inhibited. The respective chelating agent may also be comprised in the lysis solution.

Further compounds may be used to achieve the efficient lysis of the sample and/or to protect the released nucleic acids from degradation. Respective compounds that protect the released nucleic acids such as the DNA and in particular the RNA from degradation are well known in the prior art and thus, do not need a detailed description here. Respective compounds may be added separately to the whole blood sample or may be included in the lysis solution.

Specific lysis efforts might be necessary in case the isolation of a specific target pathogen nucleic acid is intended in order to ensure the efficient release of the pathogen nucleic acids. E.g. when intending to isolate nucleic acids from gram positive bacteria from a veterinary whole blood sample, specific lysis steps may be recommendable, in order to efficiently release the respective pathogen nucleic acids. Suitable lysis steps are subsequently described for some major pathogens. However, also alternative methods can be used and suitable lysis method for different pathogens are also well known in the prior art. Therefore, they are not described in detail here.

As described above, one or more of the compounds that are added for lysis of the veterinary whole blood sample may be conveniently comprised in a lysis solution. The respective lysis solution preferably comprises at least one chaotropic agent and at least one detergent as described above. Furthermore, it may comprise a chelating agent and a buffering compound. The proteolytic enzyme that is preferably added to digest the veterinary whole blood sample is preferably added separately to the whole blood sample in order to prevent degradation by the chaotropic agent that is comprised in the lysis solution.

For preparing the binding mixture, the lysed sample may then be contacted with one or more further compounds to prepare the binding conditions that allow the efficient binding of the nucleic acids to the nucleic acid binding column. Binding as used herein in particular refers to the adsorption of the nucleic acids to the nucleic acid binding solid phase. If and which compounds are added to establish the binding mixture according to the present invention also depends on the composition of the lysed sample and therefore the compounds that might have been added to achieve the lysis of the sample.

The binding mixture according to the present invention comprises at least one chaotropic agent. If no chaotropic agent was added during a separate lysis step (see above), it must be added during the preparation of the binding mixture, for example by adding a binding solution comprising a chaotropic agent. Any chaotropic agent can be used for that purpose that causes disorder in a protein or nucleic acid by, for example, but not limited to altering the secondary, tertiary or quaternary structure of a protein or a nucleic acid while leaving the primary structure intact. Preferably, a chaotropic salt is used. The chaotropic salt preferably comprises guanidinium, thiocyanate, isothiocyanate, perchlorate, trichloroacetate and/or trifluoroacetate as chaotropic ion. Preferably, the chaotropic agent is selected from the group consisting of guanidinium hydrochloride, guanidinium thiocyanate, guanidinium isothiocyanate, sodium thiocyanate, sodium iodide, sodium perchlorate, sodium trichloroacetate, sodium trifluroacetate and urea. Also mixtures of chaotropic agents can be used. In particular, guanidinium hydrochloride and/or guanidinium thiocyanate can be used as chaotropic agent. Preferably, a further amount of at least one chaotropic agent or mixture of chaotropic agents is added to prepare the binding mixture, even if a chaotropic agent was already added during the lysis of the sample. The respective binding solution may comprise the chaoptropic agent, which preferably is a chaotropic salt as mentioned above, in a concentration that lies in a range selected from about 0.1M up to the saturation limit, about 0.2M to 6M, about 0.5M to 4M, about 1M to 3.5M or about 1.5M to 3.5M. The concentration of the chaotropic agent in the binding mixture is preferably selected from about 0.2M to 6M, about 0.5M to 4M, about 1M to 3.5M or about 1M to 3M

The binding mixture according to the present invention comprises at least one alcohol. The alcohol promotes the binding of the nucleic acids to the solid phase comprised in the column. The alcohol is preferably a short chained branched or unbranched alcohol with 1 to 5 carbon atoms. Preferably, it is selected from methanol, ethanol, propanol, isopropanol and butanol. Particularly suitable are ethanol and isopropanol. The alcohol is preferably comprised in the binding mixture in a concentration selected from at least 10% v/v, at least 15% v/v and at least 25% v/v. According to one embodiment, the alcohol concentration in the binding mixture is less than 60% v/v, preferably less than 50% v/v, less than 40% v/v or less than 30% v/v. Also mixtures of alcohols can be used.

Furthermore, the binding mixture comprises at least one polyoxyethylene fatty alcohol ether. Also mixtures of polyoxyethylene fatty alcohol ethers can be used. Suitable embodiments are described above. Preferably, it is selected from the group consisting of polyoxyethylene lauryl ether, polyoxyethylene cetyl ether, polyoxyethylene stearyl ether and polyoxyethylene oleyl ether. More preferably, the polyoxyethylene fatty alcohol ether is selected from the group consisting of polyoxyethylene(4) lauryl ether, polyoxyethylene(23) lauryl ether, polyoxyethylene(2) cetyl ether, polyoxyethylene(10) cetyl ether, polyoxyethylene(20) cetyl ether, polyoxyethylene(2) stearyl ether, polyoxyethylene(10) stearyl ether, polyoxyethylene(20) stearyl ether, polyoxyethylene(2) oleyl ether, polyoxyethylene(10) oleyl ether, polyoxyethylene(20) oleyl ether and/or polyoxyethylene(100) stearyl ether. The numbers here indicate the average number of ethylene oxide units. Most preferred, the polyoxyethylene fatty alcohol ether is a polyoxyethylene cetyl ether, most preferred polyoxyethylene (20) cetyl ether. As is shown by the examples, polyoxyethylene (20) cetyl ether is specifically suitable for preventing the clogging of the column.

The polyoxyethylene fatty alcohol ether preferably is comprised in the binding mixture in a concentration selected from at least 0.5%, at least 1%, at least 2%, at least 3% and at least 5%. Preferably, the concentration lies in a range selected from about 0.5% to about 20%, about 2% to about 12%, about 3% to about 9%, preferably 4% to about 8%. These concentration ranges are particularly suitable when using polyoxyethylene (20) cetyl ether.

As discussed above, the polyoxyethylene fatty alcohol ether can be introduced into the binding mixture by the lysis solution. If no respective polyoxyethylene fatty alcohol ether has been added to achieve the lysis of the sample, it must be added during preparation of the binding mixture. However, even if a respective polyoxyethylene fatty alcohol ether was added during lysis of the sample, it is preferred that a further amount thereof is added during the preparation of the binding conditions. This can be achieved by the addition of a binding solution which comprises a respective polyoxyethylene fatty alcohol ether.

Therefore, according to a preferred embodiment, a binding solution is added to the lysed sample during preparation of the binding mixture wherein said binding solution comprises at least one chaotropic agent (or mixtures of chaotropic agents), preferably a chaotropic salt as described above, at least one polyoxyethylene fatty alcohol ether as described above and optionally a buffering substance. Suitable buffering agent include but are not limited to tris(hydroxymethyl)aminomethane (TRIS), N-(tri(hydroxymethyl)methyl)glycine (Tricine), N,N-bis(2-hydroxyethyl)glycine (BICINE), N-(2-hydroxyethyl)piperazine-N′-(2-ethanesulphonic acid) (HEPES), piperazine-1,4-bis(2-ethanesulphonic acid) (PIPES), N-cyclohexyl-2-aminoethanesulphonic acid (CHES), 2-(N-morpholino)ethanesulphonic acid (MES), 3-(N-morpholino)propanesulphonic acid (MOPS) and/or phosphate buffer. Furthermore, the binding solution may also comprise at least one alcohol as described above. However, the alcohol may also be added separately to the lysed sample to prepare the binding mixture.

The respectively prepared binding mixture is passed in step b) through a column which comprises a nucleic acid binding solid phase thereby binding the nucleic acids to the nucleic acid binding solid phase. The binding mixture may also rest first on/in the column after the binding mixture was applied thereto before it is passed through the column. As discussed above, the presence of the specific polyoxyethylene fatty alcohol ether, which preferably is a polyoxyethylene cetyl ether, more preferred a polyoxyethylene (20) cetyl ether, efficiently prevents the clogging of the nucleic acid binding solid phase comprised in the column. This was very surprising because other non-ionic detergents such as Tween 20 or Triton X-100 can not prevent the clogging.

The present invention involves the use of a nucleic acid binding solid phase that is comprised in a column. The term “column” as used herein in particular describes a container having at least two openings. Thereby, the binding mixture can pass through said column. The term “column” in particular does not imply any restrictions with respect to the shape of the container which can be e.g. round or angular and preferably is cylindrical. However, also other shapes can be used, in particular when using multi-columns. The column comprises the nucleic acid binding solid phase. Said solid phase that is comprised in said column should allow the passage of the binding mixture according to the present invention when applied to the column. This means that if e.g. a centrifuge force is applied to the column, the binding mixture is enabled to pass through the column in direction of the centrifuge force. Alternatively, a negative or positive pressure can be applied. When using a respective column based nucleic acid isolation procedure, the binding mixture is usually passed through the column, e.g. assisted by centrifugation or vacuum, and the nucleic acids bind to the comprised nucleic acid binding solid phase during said passage. The column can be used in a single format or in a multi-format. Such multi-columns having a similar format as multi-well plates and which comprise a nucleic acid binding solid phase such as a membrane, are well-known in the prior art. Preferably, the column is a spin column.

As nucleic acid binding solid phase, any solid phase can be used that is usually utilized in column based nucleic acid isolation procedures. Preferably, the nucleic acids bind to the solid phase by adsorption. The material of the nucleic acid binding solid phase that is included in the column may be made of or may comprise compounds comprising silicon dioxide including but not limited to silica, silica particles, silicon dioxide, diatomaceous earth, glass, alkylsilica, aluminum silicate, and borosilicate; nitrocellulose; diazotized paper; hydroxyapatite (also referred to as hydroxyl apatite); nylon; metal oxides; zirconia; alumina; polymeric materials, materials comprising nucleic acid binding functional groups (preferably anion exchange groups) and the like. The term solid phase is not intended to imply any limitation regarding its form or design as long as it can be comprised in a column. According to one embodiment, the surface of the solid phase such as e.g. the silica solid phase is not modified and is, e.g., not modified with functional groups. Preferably, a nucleic acid binding membrane, and thus a membrane that is capable of binding nucleic acids is used. Suitable membranes include but are not limited to hydrophilic membranes, hydrophobic membranes and membranes which bind nucleic acids via ion exchange. Examples include but are not limited to membranes comprising or consisting of silicon dioxide, silica membranes, glass fiber membranes, nylon membranes, cellulose membranes such as nitrocellulose membranes, modified cellulose membranes (e.g. acetyl- or hydroxy-), paper membranes, in particular modified papers. Preferably, the membrane is porous.

Furthermore, it is preferred to use a membrane comprising or consisting of silica. A further common nucleic acid binding solid phase comprised in a column is a fill of nucleic acid binding particles, such as metal oxide particles, in particular silica particles, or a layer of a nucleic acid binding material (e.g. a silica gel). E.g. the nucleic acid binding particles such as the silica particles can be arranged as a layer on an inert filter or membrane, thereby forming a nucleic acid binding solid phase. The problems described above may also occur with a respective nucleic acid binding solid phase. This, because a packed, thin layer of a nucleic acid binding solid phase comprised in a column (similar to a nucleic acid binding membrane) increases the risk that the binding mixture can not pass and thus increases the risk that the column is clogged. According to one embodiment, the nucleic acid binding solid phase comprised in the column has an overall height which is equal to or smaller than its width. E.g. the nucleic acid binding solid phase may be composed of a layer of a nucleic acid binding material and/or a fill, respectively layer of nucleic acid binding particles, preferably silica particles, which is applied as a small layer onto a membrane or filter.

To alleviate the passage of the binding mixture through the nucleic acid binding solid phase comprised in the column, suitable means can be used such as e.g. centrifugation or the use of a pressure difference-generating apparatus which e.g. presses the sample through the column, respectively the nucleic acid binding solid phase or sucks it through the nucleic acid binding solid phase by applying a vacuum. Respective means are well known in the prior art and thus need no further description here.

According to one embodiment, one or more washing steps are performed in step c) in order to further purify the nucleic acids. According to one embodiment, one or more washing steps are performed while the nucleic acid is bound to the solid phase. For this purpose common washing solutions may be used. According to one embodiment, the solution used for washing comprises at least one chaotropic agent and at least one alcohol. Chaotropic agents that can be used in the washing solutions include but are not limited to guanidinium hydrochloride, guanidinium thiocyanate, guanidinium isothiocyanate and sodium iodide or other chaotropic salts. As alcohol, short chained branched or unbranched alcohols with preferably one to 5 carbon atoms can be used for washing, respectively in the washing solution. Examples are methanol, ethanol, propanol, isopropanol and butanol. Preferably, isopropanol and/or ethanol are used. Preferably, the washing solution comprises at least 50% alcohol and at least 1M chaotropic salt, preferably at least 2M chaotropic salt, more preferred at least 3M chaotropic salt.

A further suitable washing solution which can be used alternatively or also in addition to the washing solutions described above comprises an alcohol and optionally a biological buffer. Preferably, isopropanol or ethanol, most preferred ethanol is used for this second washing step. Preferably, ethanol is used in a concentration of at least 50% v/v, at least 60% v/v, preferably at least 70% v/v. The biological buffer is preferably Tris.

A further suitable washing solution which can be used alternatively or optionally also in addition to the washing solutions described above comprises an alcohol but no salt. Preferably, the alcohol is comprised in a concentration of at least 50% v/v, at least 60% v/v, preferably at least 70% v/v. Preferably, the concentration lies in a range of 50% v/v to 100% v/v, more preferred 90% v/v to 100% v/v.

According to one embodiment, the nucleic acid binding solid phase comprised in the column (or a portion thereof) is directly subjected with the bound nucleic acids to an analysis such as e.g. an amplification reaction. It is well known that it is e.g. possible to directly subject a membrane or another nucleic acid binding solid phase with the bound nucleic acids into a PCR reaction. The nucleic acids are here at least partially eluted due to the PCR conditions. However, preferably, a separate elution step is performed as step d). Here, elution can be performed for example with classical elution solutions such as water, elution buffers, in particular biological buffers such as Tris or other suitable biological buffers and preferably elution solutions are used that do not interfere with the intended downstream application. Therefore, e.g. low salt solutions can be used for elution. The elution solution may comprise azide. Elution may also be assisted by heating. After elution, the eluate can also be heat denatured.

The veterinary whole blood sample may be obtained from an ungulate or a mammal with claws. Preferably it is obtained from a large domestic animal, preferably an ungulate such as cattle, sheep, goat, horse, zebu, zebra, buffalo, water buffalo, donkey, alpaca, dromedary, camel, llama, deer and pig. The animal whole blood samples may be or may have been treated after collection with EDTA, citrate or heparin as anticoagulant. Respectively treated/stabilised whole blood samples are suitable for use in the method according to the present invention and often will be the standard when processing whole blood samples. The sample can either be fresh or frozen. Preferably, 50 μl to 500 μl, preferably 50 μl to 250 μl, veterinary whole blood is used a starting material. Typically, 200 μl whole blood can be used when using the method according to the present invention. Of course, the suitable input volume also depends on the size of the column. Up to 250 μl, preferably up to 200 μl, whole blood is recommended as input volume for a standard laboratory spin column having an overall holding capacity of approx. 600 to 700 μl. However, highly elevated cell counts e.g. due to inflammatory or neoplastic diseases may strongly increase the host nucleic acid content in a sample. In this case, a reduction of sample input may improve the results in the downstream assays.

Furthermore, the method according to the present invention may also be used for other veterinary sample types such as tissue, plasma, serum, milk, urine, swabs, washes and the like. The examples demonstrate that the method according to the present invention not only allows to isolate nucleic acids efficiently from veterinary whole blood but also from other veterinary samples. It is particularly suitable for isolating nucleic acids from samples wherein a clogging risk exists due to the composition of the sample such as milk samples or certain tissues samples. Therefore, according to one embodiment, the method according to present invention is not used for isolate nucleic acids from whole animal blood but is used in order to isolate nucleic acids from milk samples or other samples, in particular samples which pose a risk of clogging the column if the polyoxyethylene fatty alcohol ether is not incorporated into the binding mixture.

The term “nucleic acid” as used herein, in particular refers to a polymer comprising ribonucleosides and/or deoxyribonucleosides that are covalently bonded, typically by phosphodiester linkages between subunits, but in some cases by phosphorothioates, methylphosphonates, and the like. Nucleic acids include, but are not limited to, gDNA; circular DNA; low molecular weight DNA, plasmid DNA; circulating DNA; hnRNA; mRNA; noncoding RNA (ncRNA), including but not limited to rRNA, tRNA, miRNA (micro RNA), siRNA (small interfering RNA), snoRNA (small nucleolar RNA), snRNA (small nuclear RNA) and stRNA (small temporal RNA); pathogen nucleic acids such as viral or bacterial nucleic acids, fragmented or degraded nucleic acids; nucleic acid obtained from subcellular organelles such as mitochondria or chloroplasts; and nucleic acid obtained from microorganisms, parasites, or DNA or RNA viruses that may be present in a biological sample. Synthetic nucleic acid sequences that may or may not include nucleotide analogs that are added or “spiked” into a biological sample are also within the scope of the invention.

The method according to the present invention is particularly suitable for isolating RNA and DNA together. However, it is also within the scope of the present invention to apply specific measures in order to isolate DNA separately from RNA or vice versa. Suitable methods include but are not limited to methods wherein a suitable solid phase is added first under conditions where mainly the non-target nucleic acids are bound to the solid phase. Suitable methods for selectively removing a non-target nucleic acid from a target nucleic acid thereby allowing for example the separation of DNA from RNA are described in EP 0 880 537 and WO 95/21849. Additionally or alternatively degrading enzymes (DNAses and RNAses) could be used.

According to one embodiment, the isolated nucleic acids comprise a pathogen nucleic acid, i.e. a nucleic acid originating from a pathogen. As is shown by the examples, the method according to the present invention is particularly suitable for isolating pathogen nucleic acids from a veterinary whole blood sample. The isolation of pathogen nucleic acids from animal whole blood is particularly difficult because respective pathogen nucleic acids are often comprised therein only in small amounts. Pathogen nucleic acids include but are not limited to nucleic acids derived from viruses, bacteria including gram negative and gram positive bacteria, parasites such as protozoan- and eukaryotic parasites. Examples of major pathogens that are important for the veterinary field include but are not limited to bovine viral diarrhea virus (BVDV), bluetongue virus (BTV), porcine circa virus (PCV), salmomella spp. and babesia spp. In particular, the present invention provides advantages for preparing nucleic acids for the analysis or detection of the presence or the amount of pathogens wherein the diagnostic method of choice is based or must be based on a nucleic acid extraction from whole blood, as is e.g. the case for the bluetongue virus.

The treatment with chemicals and proteinase K as described above is usually sufficient for a complete lysis of the sample also in case the sample comprises or is suspected of comprising gram-negative bacteria. However, depending on the type of target pathogen, specific lysis measures are recommended in order to ensure the effective lysis of the sample and the release of the potentially comprised pathogen nucleic acids. Therefore, it might be advisable to perform a specific pre-treatment protocol in order to ensure an efficient lysis. E.g., the cell wall of gram-positive bacteria should be disrupted usually by using additional methods. For maximal lysis efficiency when intending to isolate nucleic acids from such bacteria from the whole blood sample, it is recommended to assist the lysis by mechanical disruption, for example using glass beads. Furthermore, the samples may be treated with an antifoaming agent and an anionic detergent such as preferably SDS. Therefore, according to one embodiment, the whole blood sample is optionally during mechanical disruption (see above) treated with an antifoaming agent and a lysis solution comprising an anionic detergent, preferably SDS in a concentration of at least 1%, preferably in a concentration of 2% to 15%, 2% to 10%, most preferably between 2 and 5%. The concentration of the anionic detergent, preferably SDS, in said (pre)lysis mixture preferably lies in a range from 0.3% to 5%. Said lysis solution may additionally comprise a chelating agent and a salt. The pH is preferably above 8. Then, the respective mixture additionally comprising for example glass beads for supporting the mechanical disruption is vortexed for approximately 5 to 15 minutes. The supernatant is obtained and then subjected to regular the lysis procedure as described above, wherein preferably a chaotropic agent, a non-ionic detergent and a proteolytic enzyme is added. Details of a suitable lysis protocol are described above.

The nucleic acids extracted with the method according to the present invention are free of proteins, nucleases and other impurities and are therefore ready for use in downstream applications such as in methods for detecting the presence or absence of a specific target nucleic acid or the quantification thereof, or for determining the genotype of a target. Basically, the nucleic acids isolated from the veterinary whole blood sample can be used in any standard method that involves the use of respective nucleic acids, including vertebrae genomic DNA, including but not limited to pathogen detection and genotyping, e.g. for breeding purposes.

The analysis/further processing of the isolated nucleic acids can be performed using any nucleic acid analysis/processing method including, but not limited to amplification technologies, polymerase chain reaction (PCR), isothermal amplification, reverse transcription polymerase chain reaction (RT-PCR), quantitative real time polymerase chain reaction (Q-PCR), digital PCR, gel electrophoresis, capillary electrophoresis, mass spectrometry, fluorescence detection, ultraviolet spectrometry, hybridization assays, DNA or RNA sequencing, restriction analysis, reverse transcription, NASBA, allele specific polymerase chain reaction, polymerase cycling assembly (PCA), asymmetric polymerase chain reaction, linear after the exponential polymerase chain reaction (LATE-PCR), helicase-dependent amplification (HDA), hot-start polymerase chain reaction, intersequence-specific polymerase chain reaction (ISSR), inverse polymerase chain reaction, ligation mediated polymerase chain reaction, methylation specific polymerase chain reaction (MSP), multiplex polymerase chain reaction, nested polymerase chain reaction, solid phase polymerase chain reaction, or any combination thereof. Respective technologies are well-known to the skilled person and thus, do not need further description here.

Furthermore, the present invention provides a method for isolating pathogen acids from a veterinary whole blood sample, wherein a method for isolating nucleic acids as described above is performed. As is shown by the examples, the respective method is particularly suitable for isolating pathogen nucleic acids from a veterinary whole blood sample.

Furthermore, the present invention pertains to a method for detecting the presence or absence of at least one target pathogen nucleic acid in a veterinary whole blood sample, comprising performing the method according to the present invention according to the first aspect and detecting the presence or absence of at least one pathogen target nucleic acid in the isolated nucleic acids. Preferably, the target pathogen nucleic acid is detected using an amplification method, preferably by polymerase chain reaction. In case a pathogen RNA supposed to be detected, a RT-PCR is preferably performed. The detection also encompasses the quantification of pathogen nucleic acids. Typical pathogens are described above, it is referred to the respective disclosure.

The invention is now illustrated by various examples, which are non-limiting to the scope of the present invention.

### EXAMPLES

The inventors have tested and compared numerous nucleic acid isolation methods which all use a chaotropic salt and alcohol (and predominantly also a non-ionic detergent) in the binding mixture for their ability to isolate nucleic acids from veterinary whole blood samples. It was found, that with standard membrane based nucleic acid purification procedures, clogging of the column always occurred even though different chaotropic salts (for example guanidine hydrochloride or guanidine thiocyanate), different chaotropic salt concentrations (for example 1.9 to 2.6M) and/or different alcohol concentrations (e.g. 10 to 45%) were used in the binding mixture. Furthermore, also non-ionic detergents (e.g. Tween 20 or Triton X100) that are used in these prior art protocols could not prevent the clogging of the column. Therefore, even though these methods work very well for isolating nucleic acids from various veterinary sample types (such as plasma, urine and tissue), they were inappropriate to isolate nucleic acids from veterinary whole blood samples because the columns clog. Therefore, it was very surprising that the observed clogging of the column can be prevented by basically using the standard nucleic acid isolation protocols wherein, however, a specific detergent is included in the binding mixture. It was found that the clogging of the column can be efficiently prevented when incorporating into the binding mixture at least one polyoxyethylene fatty alcohol ether. Thereby, membrane clogging can be efficiently prevented when isolating nucleic acids from animal whole blood. Clogging of the column can be prevented when using a respective binding mixture. Furthermore, the examples show that different lysis methods can be used in order to provide the lysed sample. Furthermore, the examples demonstrate that the clogging of the column is also prevented when using different concentrations of alcohol(s), chaotropic salt(s) and polyoxyethylene fatty alcohol ether.

Therefore, the incorporation of the respective specific detergent, which preferably is a polyoxyethylene cetyl ether, more preferred polyoxyethylene(20) cetyl ether, in the binding step greatly reduces the risk that the membrane clogs when isolating nucleic acids from animal whole blood.

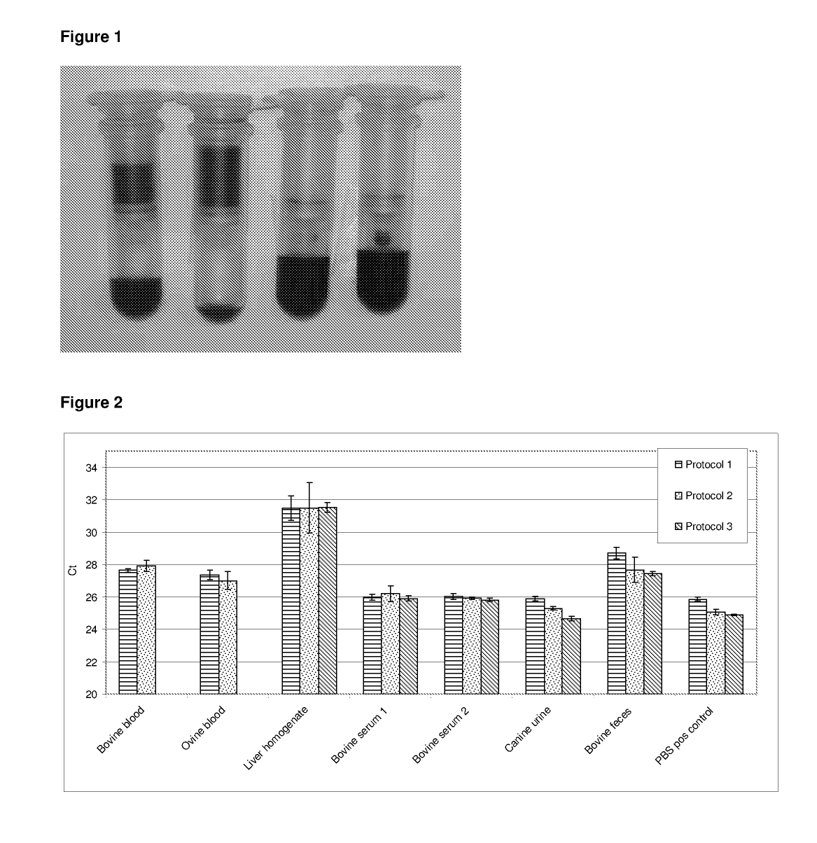

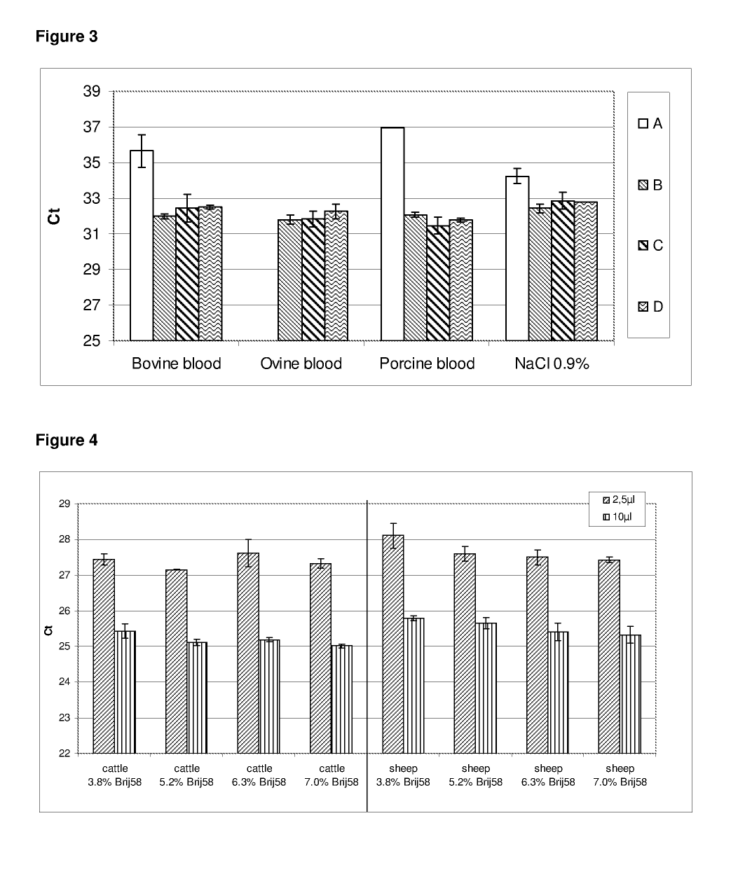

The advantageous effects of the method according to the present invention compared to prior art methods are shown in FIG. 1. FIG. 1 shows the clogging that occurs when trying to isolate nucleic acids from bovine whole blood with standard methods that employ a binding buffer containing guanidine hydrochloride and Tween 20 in a concentration of approx. 6.5% (see tube 1+2). The binding mixture can not pass through the column because clogging occurs. No clogging occurs if polyoxyethylene(20) cetyl ether is incorporated in the binding mixture in a concentration of approx. 10% (see tubes 3+4).

### 1. Example 1

Different veterinary samples (and as a positive control PBS buffer) were spiked with BVDV virus particles and the nucleic acids were isolated with different protocols which use different binding conditions. In all protocols, proteinase K (20 μl) was added for lysis.

In protocol 1, lysis was performed by adding a lysis buffer comprising polyoxyethylene(20) cetyl ether and GTC. Afterwards, a binding buffer comprising GTC, isopropanol and polyoxyethylene(20) cetyl ether was added to the lysed sample. The resulting binding mixture comprised 2.31M GTC, 13% isopropanol and 6.5% polyoxyethylene(20) cetyl ether.

In protocol 2, lysis was performed by adding a lysis buffer comprising guanidine hydrochloride and a mixture of non-ionic detergents (Tween 20 and Triton X-100). Afterwards, a binding buffer comprising GTC, isopropanol and polyoxyethylene(20) cetyl ether was added to the lysed sample. The resulting binding mixture comprised the chaotropic salts GTC and GuHCL in an overall concentration of 2.3M, 22% isopropanol and 6.7% polyoxyethylene(20) cetyl ether.

In protocol 3, lysis was performed by adding a lysis buffer comprising guanidine hydrochloride and Triton X-100. Afterwards, a binding buffer comprising GTC and ethanol was added to the lysed sample. The resulting binding mixture comprised the chaotropic salts GTC and GuHCL in an overall concentration of 2M, 33% ethanol and Triton X-100.

5 μl of the nucleic acids isolated with each protocol were used in a subsequent RT-PCR for detecting BVDV RNA in the isolated nucleic acids. Lower Ct values obtained in such an assay indicate that more BVDV RNA was present in the reaction and hence, that the BVDV RNA was isolated with better yield. The results are shown in FIG. 2. Protocols 1 to 3 achieved rather identical Ct values for PBS positive control sample, serum, urine and feces. This shows that all protocols 1 to 3 work equally well when isolating nucleic acids from said veterinary samples. However, BVDV RNA could not be detected in the nucleic acids that were isolated from different veterinary whole blood samples (bovine and ovine blood) using protocol 3. With protocol 3, the columns clogged. Therefore, it was not possible to isolate the nucleic acids from animal whole blood when using protocol 3 comprising Triton X-100 as non-ionic detergent in the binding mixture. However, nucleic acids could be efficiently isolated from said veterinary whole blood samples using protocols 1 and 2 and hence, the method according to the present invention.

### 2. Example 2

200 μl of whole blood samples from different species (and as a positive control a 0.9% sodium chloride solution) were spiked with BVDV particles. Said samples were processed with either a standard method employing a binding mixture containing guanidine hydrochloride (GuHCL) and Tween 20 or different variants of the teachings of the present invention employing different concentrations of guanidine thiocyanate (GTC) and polyoxyethylene(20) cetyl ether in the binding mixture. Lysis was supported by the addition of proteinase K:

Method A: 1.81M GuHCl, 32.2% EtOH, 6.5% Tween 20 in the binding mixture

Method B: 1.9 M GTC, 24% isopropanol, 8.5% polyoxyethylene(20) cetyl ether in the binding mixture

Method C: 1.5M GTC, 17% isopropanol, 7.6% polyoxyethylene(20) cetyl ether, 0.3% SDS in the binding mixture

Method D: 1.5M GTC, 19% isopropanol, 6.8% polyoxyethylene(20) cetyl ether, 0.3% SDS in the binding mixture

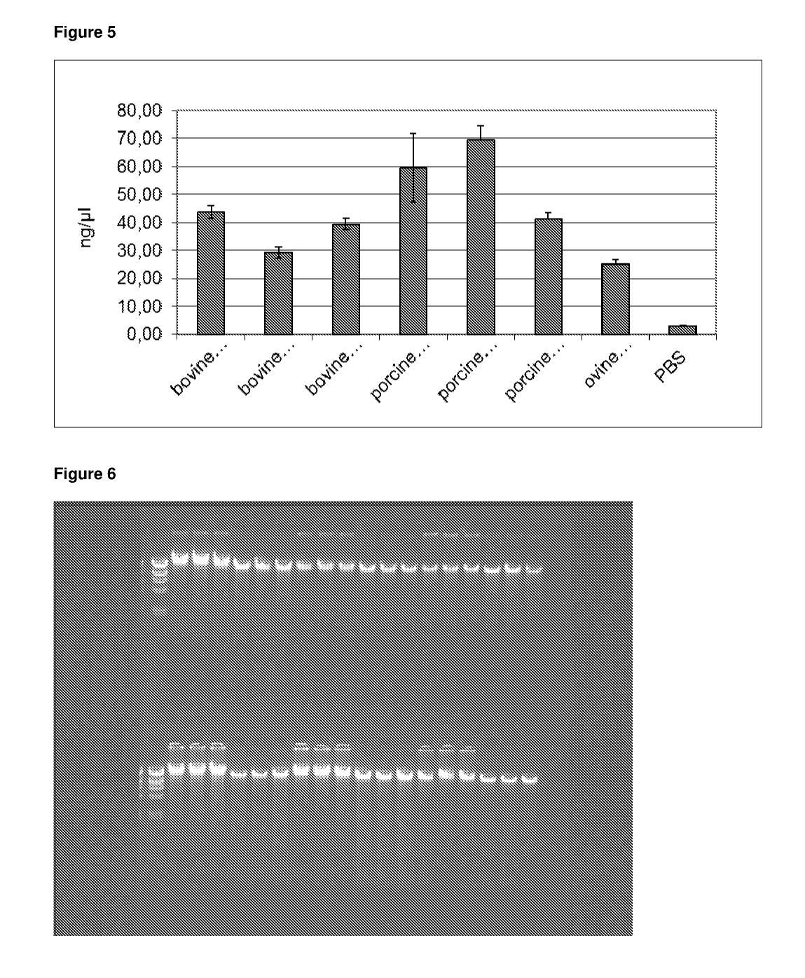

Processing was done on a QlAamp 96 plate using a vacuum device. When using method A, clogging of the column did occur,. No clogging was observed when using methods B, C and D. In a subsequent RT-PCR for detecting BVDV RNA comprised in the isolated nucleic acids, lower Ct values were achieved with methods B, C and D than with method A, where even drop outs of data points for sheep and pig blood occurred. Lower Ct values indicate that more BVDV RNA was present in the reaction and hence, that the BVDV RNA was isolated with better yield. The results are shown in FIG. 5.

### 3. Example 3

Cattle and sheep blood was spiked with BHV1 virus particles and processed using different variants of the method according to the present invention. The samples were lysed using proteinase K and a lysis solution comprising a mixture of non-ionic detergents (Tween 20 and Triton X-100) and a chaotropic salt (GuHCL). For binding, different volumes of a binding solution comprising a chaotropic salt (GTC), isopropanol and polyoxyethylene(20) cetyl ether was added. Thereby, different binding mixtures were obtained having different concentrations of chaotropic salt (2.14M to 2.36M), alcohol (12.8% to 23.4%) and polyoxyethylene(20) cetyl ether (3.8% to 7%). Clogging could be efficiently prevented using the different concentrations of polyoxyethylene(20) cetyl ether. All different variants of the method according to the present invention were able to isolate the nucleic acids from the tested whole blood samples as is shown by the results presented as FIG. 6. Lack of inhibition of the PCR reaction is shown by a ct difference of approx. 2 between 2.5 μl and 10 μl template volume. Thus, the method according to the present invention works with different concentrations of chaotropic salts, alcohol and polyoxyethylene(20) cetyl ether.

4. Example 4

FIGS. 5 and 6 furthermore demonstrate that the method according to the present invention is suitable to isolate cellular nucleic acids, here genomic DNA, from different animal whole blood samples. FIGS. 5 and 6 show a photometric measurement and an ethitiumbromide stained agarose gel of 6 different animal blood samples processed with protocol 2 of example 1. The gel shows a comparison to an automated paramagnetic silica-particles based DNA isolation procedure (QIAsymphony DNA blood). Three replicates for each sample are shown.

5. Example 5

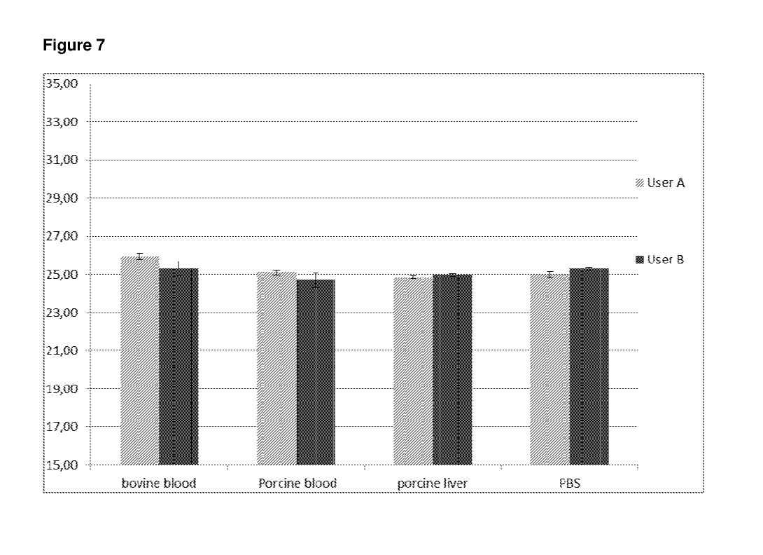

It is also important that a nucleic acid isolation protocol works reliably, and hence can be used equally effective by different users. To test said inter-user consistency, different samples were spiked with S. enterica. Bacterial DNA was extracted using protocol 2 of example 1. The same samples were processed in parallel by different users. The results are shown in FIG. 7. A high inter-user consistency is achieved with the method according to the present invention what is highly important for the medical/diagnostic field.

### 6. Example 6

According to this protocol according to the present invention, 20 μl proteinase K is pipetted into a 2 ml microcentrifuge tube. 200 μl whole blood sample is added to the proteinase K. Afterwards, a lysis buffer is added, which comprises guanidine hydrochloride in a concentration above 5M and a mixture of non-ionic detergents in a concentration around 15% (Tween 20 and Triton X-100). To ensure efficient lysis, the sample is mixed with the lysis buffer to yield a homogenous solution. Thereby, a lysis mixture is obtained comprising approx. 2M GuHCL, approx. 5% non-ionic detergents (Tween 20 and Triton X-100). If the isolation of target nucleic acids from gram-positive bacteria is intended, it is recommended to pre-treat the samples before the proteinase K treatment with a lysis buffer comprising an anionic detergent, preferably SDS (e.g. the buffer ATL (QIAGEN) could be used).

The respective mixture is incubated at 20 to 25° C. for 15 minutes to lyse the sample. The tubes were afterwards briefly centrifuged to remove drops from the inside of the lid. Afterwards, 350 μl binding buffer was added to the sample. The respective buffer comprises a polyoxyethylene (20) cethylether, a chaotropic salt (GTC) and isopropanol. The caps are closed and mixed thoroughly by pulse-vortexing. Thereby, a binding mixture is obtained comprising 2.3M chaotropic salt (GuHCl and GTC), 21% isopropanol and 6.3% polyoxyethylene (20) cethylether.

The respectively prepared binding mixture is placed into a QIAamp Mini column (comprising a silica membrane) in a 2 ml collection tube without wetting the rim. The cap is closed, and centrifuged at 8000 rpm for one minute. The QIAamp Mini column is placed into a clean 2 ml collection tube and the collection tube containing the filtrate is discarded.

Afterwards, 600 μl washing buffer AW1 (QIAGEN) is added without wetting the rim. After centrifugation at 8000 rpm for one minute, the QIAamp Mini column is placed into a clean 2 ml collection tube and the tube containing the filtrate is discarded. Afterwards, 600 μl washing buffer AW2 (QIAGEN) is added without wetting the rim. After centrifugation for one minute at 8000 rpm, the QIAamp Mini column is placed into a clean 2 ml collection tube and the filtrate containing tube is discarded. Afterwards the membrane is dried by centrifugation at full speed (14.000 rpm) for two minutes.

The QIAamp Mini column is placed into a clean 1.5 ml micro centrifuged tube and the collection tube containing the filtrate is discarded. For elution, 50 to 150 μl buffer AVE (QIAGEN) is added to the centre of the membrane. The cap is closed and incubated at room temperature for one minute. After centrifugation at full speed (14.000 rpm) for one minute, the eluate can be collected.

1.-15. (canceled)

16. A method for isolating nucleic acids from a veterinary whole blood sample, comprising:
a) preparing a binding mixture comprising
a lysed veterinary whole blood sample,
at least one chaotropic agent,
at least one alcohol, and
at least one polyoxyethylene fatty alcohol ether;
b) passing the binding mixture through a column comprising a nucleic acid binding solid phase, thereby binding the nucleic acids to the nucleic acid binding solid phase;
c) optionally washing the nucleic acids while being bound to the solid phase; and
d) optionally eluting the nucleic acids from the solid phase.

17. The method according to claim 16, wherein
i) the polyoxyethylene fatty alcohol ether is selected from the group consisting of polyoxyethylene lauryl ether, polyoxyethylene cetyl ether, polyoxyethylene stearyl ether and polyoxyethylene oleyl ether;
ii) the polyoxyethylene fatty alcohol ether is selected from the group comprising polyoxyethylene(4) lauryl ether,polyoxyethylene(23) lauryl ether, polyoxyethylene(2) cetyl ether, polyoxyethylene(10) cetyl ether, polyoxythylene(20) cetyl ether, polyoxyethylene(2) stearyl ether, polyoxyethylene(10) stearyl ether, polyoxyethylene(20) stearyl ether, polyoxyethylene(2) oleyl ether, polyoxyethylene(10) oleyl ether, polyoxyethylene(20) oleyl ether and polyoxyethylene(100) stearyl ether;
iii) the polyoxyethylene fatty alcohol ether is a polyoxyethylene cetyl ether; and/or
iv) the polyoxyethylene fatty alcohol ether is polyoxyethylene(20) cetyl ether.

18. The method according to claim 16, wherein the polyoxyethylene fatty alcohol ether is comprised in the binding mixture at a concentration in a range selected from about 0.5% to about 20%, about 2% to about 15%, and about 3% to about 12%.

19. The method according to claim 16, wherein the polyoxyethylene fatty alcohol ether is comprised in the binding mixture at a concentration in the range of 4% to about 10%.

20. The method according to claim 16, wherein the chaotropic agent has one or more of the following characteristics:
i) it is a chaotropic salt;
ii) it is a chaotropic salt selected from the group consisting of guanidinium hydrochloride, guanidinium thiocyanate, guanidinium isothiocyanate, sodium thiocyanate, sodium iodide, sodium perchlorate, sodium trichloroacetate, and sodium trifluroacetate; and/or
iii) it is comprised in the binding mixture at a concentration in a range selected from about 0.1M to 7M, about 0.2M to 6M, about 0.5M to 4M, and about 0.5 to 3M.

21. The method according to claim 16, wherein the at least one alcohol comprised in the binding mixture has one or more of the following characteristics:
i) it is a short chained branched or unbranched alcohol with one to 5 carbon atoms;
ii) it is selected from methanol, ethanol, propanol, isopropanol, and butanol; and
iii) the alcohol is comprised in the binding mixture in a concentration selected from at least 10%, at least 15%, and at least 20%.

22. The method according to claim 16, wherein the isolated nucleic acids comprise a pathogen nucleic acid.

23. The method according to claim 22, wherein the pathogen nucleic acid is obtained from a pathogen selected from the group consisting of viruses, bacteria, and parasites.

24. The method according to claim 16, further comprising determining the presence or absence of a pathogen nucleic acid in the isolated nucleic acids.

25. The method according to claim 16, wherein the preparation of the binding mixture in step a) comprises:
i) lysing a veterinary whole blood sample; and
ii) adding to the lysed sample one or more of the following additives:
at least one chaotropic agent,
at least one alcohol, and
at least one polyoxyethylene fatty alcohol ether,
thereby preparing the binding mixture.

26. The method according to claim 25, wherein the one or more of the additives are added in form of a binding solution.

27. The method according to claim 16, wherein the veterinary whole blood sample is stabilised.

28. The method according to claim 27, wherein the veterinary whole blood sample is stabilized by the use of an antigoagulant.

29. The method according to claim 16, wherein the lysis of the sample involves one or more of the following:
i) the addition of at least one proteolytic enzyme;
ii) the addition of at least one chaotropic agent;
iii) the addition of at least one detergent;
iv) the addition of at least one chelating agent;
v) heating, and/or
vi) shaking

30. The method according to claim 29, wherein the lysis of the sample involves the addition of a lysis solution comprising at least one chaotropic agent and at least one detergent, and the addition of at least one proteolytic enzyme.

31. The method according to claim 16, wherein the veterinary whole blood sample is obtained from a large domestic animal selected from cattle, sheep, goat, donkey, horse and pig.

32. The method according to claim 29, wherein the chelating agent is selected from diethylenetriaminepentaacetic acid (DTPA), ethylenedinitrilotetraacetic acid (EDTA), ethylene glycol tetraacetic acid (EGTA), and N,N-bis(carboxymethyl)glycine (NTA).

33. The method according to claim 16, wherein the binding mixture is prepared by adding a binding solution to the lysed sample, wherein said binding solution has one or more of the following characteristics:
i) it comprises at least one chaotropic salt at a concentration selected from about 0.1M to 7M, about 0.2M to 6M, about 0.5M to 4M, and about 0.5 to 3M;
ii) it comprises at least one polyoxyethylene fatty alcohol ether selected from the group consisting of polyoxyethylene lauryl ether, polyoxyethylene cetyl ether, polyoxyethylene stearyl ether and/or polyoxyethylene oleyl ether at a concentration selected from about 0.5% to about 20%, about 2% to about 15%, and about 3% to about 12%;
iii) it comprises at least one buffering agent; and/or
iv) it comprises a short chained branched or unbranched alcohol with one to 5 carbon atoms.

34. The method according to claim 33, wherein the binding solution has at least two of characteristics i) to iv).

35. The method of claim 33, wherein in characteristic ii), the at least one polyoxyethylene fatty alcohol ether is at a concentration in a range of 4% to about 8%.
